(19) United States
(12) Patent Application Publication
(10) Pub. No.: US 2023/0309878 A1
(43) Pub. Date: Oct. 5, 2023

(54) Device for Manually Locking an Inserted Line Draw Device to Prevent Premature Retraction

(71) Applicant: Becton, Dickinson and Company, Franklin Lakes, NJ (US)

(72) Inventors: Jonathan Karl Burkholz, Salt Lake City, UT (US); Yiping Ma, Layton, UT (US)

(21) Appl. No.: 18/127,934

(22) Filed: Mar. 29, 2023

Related U.S. Application Data
(60) Provisional application No. 63/325,987, filed on Mar. 31, 2022.

(51) Int. Cl. A61B 5/15

(52) U.S. Cl. CPC A61B 5/150992; A61B 5/15003; A61M 2025/0006

(57) ABSTRACT

A fluid transfer device for needle-free delivery of a probe to a patient's vascular system, the device including an introducer body and an actuator. The actuator is linearly displaceable along at least a portion of the introducer body, and the actuator is operably coupled to the probe to advance and retract the probe based on direction of displacement of the actuator. The device also includes a connector member positioned proximate a distal end portion of the introducer body and configured for releasable connection to at least one surface of an access connector. The device also includes a locking member configured to prevent disconnection of the connector member from the access connector when the actuator is advanced to a first position, and to allow disconnection of the connector member when the actuator is retracted to a second position along the introducer body.

### CROSS-REFERENCE TO RELATED APPLICATION

The present application claims priority to U.S. Provisional Application Ser. No. 63/325,987, entitled “Device for Manually Locking an Inserted Line Draw Device to Prevent Premature Retraction”, filed Mar. 31, 2022, the entire disclosure of which is hereby incorporated by reference in its entirety.

### BACKGROUND OF THE INVENTION

### Field of the Invention

The present disclosure generally relates to fluid transfer devices and related assemblies, systems, and methods for use in blood collection via an existing vascular access device (VAD) such as, e.g., a peripheral intravenous catheter (PIVC). Specifically, the fluid transfer devices of the present disclosure are configured to prevent premature retraction and/or removal of the device.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (“PIVC”). The over-the-needle catheter may be mounted over an introducer needle having a sharp distal tip. The catheter and the introducer needle may be assembled so that the distal tip of the introducer needle extends beyond the distal tip of the catheter with the bevel of the needle facing up away from a skin surface of the patient. The catheter and introducer needle are generally inserted at a shallow angle through the skin into the vasculature of the patient. In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using a peripheral IV catheter may be difficult for several reasons, particularly when an indwelling time of the catheter is more than one day. For example, when the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, catheters may often be used for acquiring a blood sample at a time of catheter placement but are much less frequently used for acquiring a blood sample during the catheter dwell period.

Accordingly, fluid transfer devices have been developed to collect blood samples through an existing PIVC. Such “line draw” fluid transfer devices attach to the PIVC and include a flexible probe that is advanced through the PIVC, beyond the catheter tip, and into a vessel to collect a blood sample. After blood collection, the fluid transfer device is removed from the PIVC and discarded. One example of such a fluid transfer device is known as PIVO™ from Velano Vascular, Inc. Furthermore, U.S. Pat. No. 11,090,461, which is hereby incorporated by reference in its entirety, also discloses such a device. FIG. 1 of the present application illustrates a fluid transfer device 200 similar to that shown and described in U.S. Pat. No. 11,090,461. Fluid transfer device 200 includes an introducer 210, a connector member 240, a secondary catheter 265, and an actuator 270. The introducer 210 includes a proximal end portion 211 and a distal end portion 212, with the connector member 240 being located adjacent the distal end portion 212. The secondary catheter 265 includes the proximal end portion 266 which is coupled to and/or otherwise includes a coupler 269. The coupler 269 is configured to physically and fluidically couple the secondary catheter 265 to any suitable device such as, for example, a fluid reservoir, fluid source, syringe, evacuated container holder (e.g., having a sheathed needle or configured to be coupled to a sheathed needle), pump, and/or the like.

A user may manipulate the fluid transfer device 200 to couple the connector member 240 to, e.g., the catheter adapter of an integrated intravenous catheter. For example, the user can exert a force sufficient to pivot the first and second clip arms of the connector member 240 such that a portion of the catheter adapter can be inserted into the space defined between the arms of the connector member 240 and a distal introducer portion 242 extending distally from the introducer 210. The user can then linearly displace the actuator 270 distally along the introducer 210 in order to advance the flexible probe through the distal introducer portion 242. The user may also linearly displace the actuator 270 proximally along the introducer to retract the flexible probe.

Typically, the flexible probe is retracted into the fluid transfer device 200 after blood collection, at which point the connector member 240 may be decoupled from the catheter adapter by pinching proximal clip portions to release the “alligator clip” distal connection of connector member 240 from the adapter. However, in some instances, the user may decouple the connector member 240 from the catheter adapter and retract the distal introducer portion 242 while the probe is still in its advanced state beyond the distal introducer portion 242 and within at least the catheter adapter. Such premature retraction and/or removal of the fluid transfer device 200 from the catheter adapter may result in undesirable exposure to blood or other bodily fluids, and/or may result in damage to the probe.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

In accordance with an aspect of the present disclosure, a fluid transfer device for delivery of a probe to a patient's vascular system is provided. The fluid transfer device includes an introducer body, a distal introducer portion positioned at a distal end portion of the introducer body and configured to penetrate an access connector of a vascular access device, and an actuator, wherein the actuator is linearly displaceable along at least a portion of the introducer body, and wherein the actuator is operably coupled to the probe to advance and retract the probe through the distal introducer portion based on direction of displacement of the actuator. The fluid transfer device also includes a connector member positioned proximate the distal introducer portion and configured for releasable connection to at least one surface of the access connector, wherein the connector member includes a pair of opposing distal clip portions and a pair of opposing proximal clip portions. Additionally, the fluid transfer device includes a locking member, wherein the locking member is linearly displaceable along at least a portion of the introducer body and is configured to prevent actuation of at least one of the pair of opposing proximal clip portions when the actuator is displaced at least partially toward first position and to allow actuation of at least one of the pair of opposing proximal clip portions when the actuator is displaced at least partially toward a second position.

In some embodiments, the locking member includes a single locking arm member, and the single locking arm member is configured to block actuation of a single one of the pair of opposing proximal clip portions of the connector member.

In some embodiments, the single locking arm member extends distally from a base portion, and the base portion is configured to be slidable along at least a portion of the introducer body.

In some embodiments, the locking member includes a pair of opposing arm members, and the pair of opposing arm members are configured to block actuation of the pair of opposing proximal clip portions of the connector member.

In some embodiments, the pair of opposing arm members extend distally from a base portion, and the base portion is configured to be slidable along at least a portion of the introducer body.

In some embodiments, the actuator is releasably engagable with the base portion.

In some embodiments, the locking member includes a biasing member configured to bias the locking member in one direction.

In some embodiments, the fluid transfer device further includes a holding tab, wherein the holding tab is configured to hold the locking member proximally away from the connector member.

In some embodiments, the holding tab is configured to be displaced by the actuator upon contact with the actuator, and displacement of the holding tab causes distal displacement of the locking member toward the second position under force of the biasing member.

In some embodiments, the locking member includes at least one arm member and an elongated tether portion extending proximally from the at least one arm member, wherein the at least one arm member is configured to block actuation of at least one of the pair of opposing proximal clip portions of the connector member.

In some embodiments, the actuator is configured for releasable engagement with at least a portion of the elongated tether portion.

In some embodiments, the locking member is manually displaceable by a user.

According to another aspect of the present disclosure, a fluid transfer device for needle-free delivery of a probe to a patient's vascular system is disclosed. The fluid transfer device includes an introducer body having a distal end portion and a proximal end portion, and an actuator, wherein the actuator is linearly displaceable along at least a portion of the introducer body, and wherein the actuator is operably coupled to the probe to advance and retract the probe based on direction of displacement of the actuator. The fluid transfer device also includes a connector member positioned proximate the distal end portion of the introducer body and configured for releasable connection to at least one surface of an access connector, and a locking member, wherein the locking member is configured to prevent disconnection of the connector member from the access connector when the actuator is advanced to a first position along the introducer body and to allow disconnection of the connector member when the actuator is retracted to a second position along the introducer body.

In some embodiments, the locking member includes a single locking arm member, and the single locking arm member is configured to block actuation of the connector member.

In some embodiments, the locking member includes a pair of opposing arm members, and the pair of opposing arm members are configured to block actuation of the connector member.

In some embodiments, the locking member includes at least one arm member and an elongated tether portion extending proximally from the at least one arm member, wherein the at least one arm member is configured to block actuation of the connector member.

In some embodiments, the actuator is configured for releasable engagement with at least a portion of the elongated tether portion.

Accordingly to another aspect of the present disclosure, a fluid transfer device for needle-free delivery of a probe to a patient's vascular system is disclosed. The fluid transfer device includes an introducer body having a distal end portion and a proximal end portion, and an actuator, wherein the actuator is linearly displaceable along at least a portion of the introducer body, and wherein the actuator is operably coupled to the probe to advance and retract the probe based on direction of displacement of the actuator. The fluid transfer device also includes a connector member positioned proximate the distal end portion of the introducer body and configured for releasable connection to at least one surface of an access connector, and a locking member, wherein the locking member includes at least one arm member and an elongated tether portion extending proximally from the at least one arm member, wherein the at least one arm member is configured to selectively block actuation of the connector member based upon the position of the actuator along the introducer body.

In some embodiments, the actuator is configured for releasable engagement with at least a portion of the elongated tether portion.

In some embodiments, the connector member includes a pair of opposing distal clip portions and a pair of opposing proximal clip portions, and the at least one arm member of the locking member is configured to block actuation of at least one of the pair of opposing proximal clip portions.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1 is a perspective view of a prior art fluid transfer device;

FIG. 2 is a top plan view of a fluid transfer device in a first configuration in accordance with an aspect of the present disclosure;

FIG. 3 is a top plan view of the fluid transfer device of FIG. 2 in a second configuration;

FIG. 4 is a top plan view of the fluid transfer device of FIG. 2 in a third configuration;

FIG. 5 is a top plan view of a locking member for use with the fluid transfer device of FIG. 2;

FIG. 6 is an end view of the locking member of FIG. 5;

FIG. 7 is a top plan view of a fluid transfer device in first and second configurations in accordance with another aspect of the present disclosure;

FIG. 8 is a top plan view of a fluid transfer device in a first configuration in accordance with another aspect of the present disclosure;

FIG. 9 is a top plan view of the fluid transfer device of FIG. 8 in a second configuration;

FIG. 10 is a top plan view of the fluid transfer device of FIG. 8 in a third configuration;

FIG. 11 is an end view of a locking member for use with the fluid transfer device of FIG. 8;

FIG. 12 is a top plan view of a fluid transfer device in a first configuration in accordance with another aspect of the present disclosure;

FIG. 13 is a top plan view of the fluid transfer device of FIG. 12 in a second configuration;

FIG. 14 is a top plan view of the fluid transfer device of FIG. 12 in a third configuration;

FIG. 15 is a top plan view of a locking member for use with the fluid transfer device of

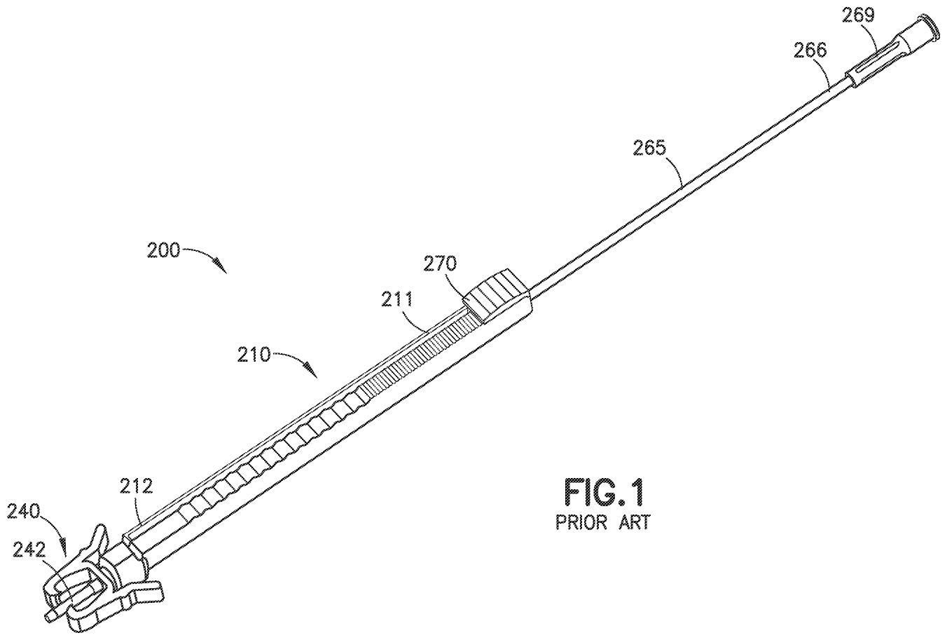

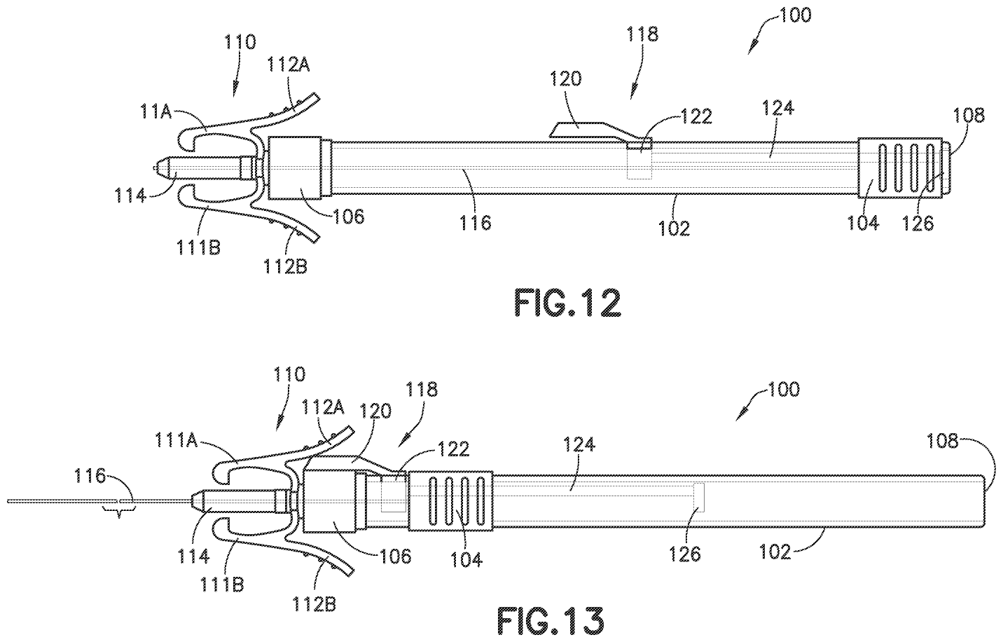

FIG. 12;

FIG. 16 is a top plan view of a fluid transfer device in a first configuration in accordance with another aspect of the present disclosure;

FIG. 17 is a top plan view of the fluid transfer device of FIG. 16 in a second configuration; and

FIG. 18 is a top plan view of the fluid transfer device of FIG. 16 in a third configuration.

### DESCRIPTION OF EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a fluid transfer device in preparation for or during use. Similarly, in this application, the terms “in the distal direction” and “distally” mean in the direction toward the connector portion of the fluid transfer device, and the terms “in the proximal direction” and “proximally” mean in the direction opposite the direction of the connector.

While not shown or described herein, it is to be understood that the fluid transfer devices described below may be utilized for blood draw from any suitable vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

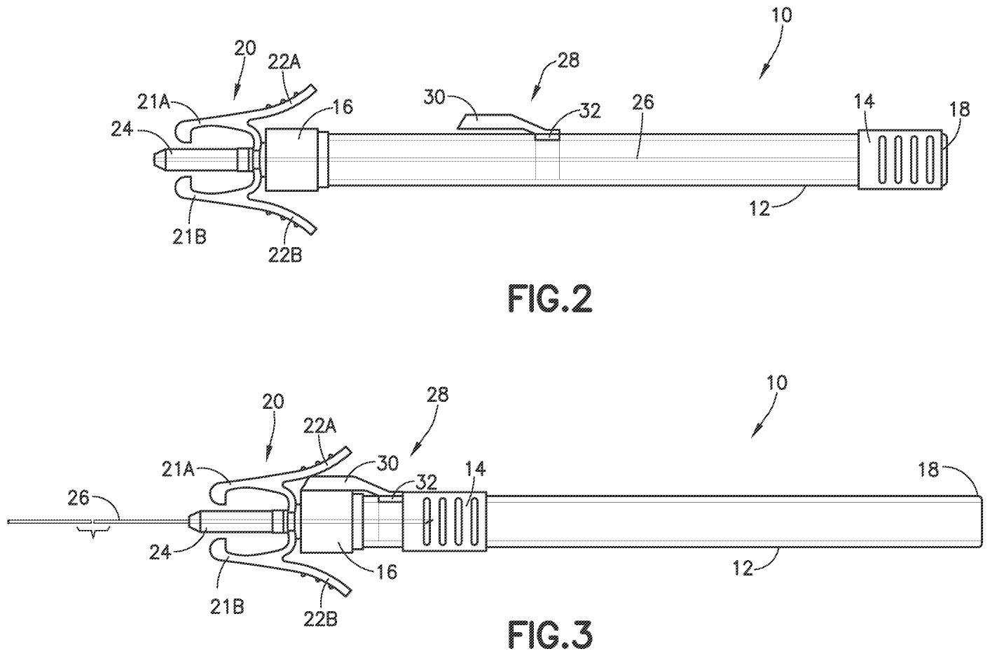

Referring to FIGS. 2-4, a fluid transfer device 10 in accordance with an aspect of the present disclosure is illustrated. The fluid transfer device 10 includes an introducer body 12 and an actuator 14. The actuator 14 is configured to be linearly displaceable by a user along a track or other feature of the introducer body 12 between a proximal end portion 18 and a distal end portion 16 of the introducer body 12. The actuator 14 is operably coupled to an elongated, flexible probe 26 to advance and retract the probe 26 through a distal introducer portion 24 located near the distal end portion 16 of the introducer body 12. That is, displacement of the actuator 14 in first direction advances the probe 26 through the distal introducer portion 24 such that the probe 26 can enter the patient's vasculature when fluid transfer device 10 is coupled to a vascular access device. Conversely, displacement of the actuator 14 in a second, opposite direction retracts the probe 26 through the distal introducer portion 24, ultimately removing the probe 26 from the patient's vasculature.

In some embodiments, the probe 26 is configured as a guidewire (e.g., a nickel titanium wire). However, in other embodiments, probe 26 may be configured as any flexible member or instrument capable of being advanced through a catheter such as, e.g., tubing, a secondary catheter, one or more sensors, obturators, or any combination thereof. Furthermore, in some embodiments, the distal introducer portion 24 is configured as a blunt plastic cannula usable with a needle-free connector of a vascular access device. However, in other embodiments, distal introducer portion 24 may be configured as a male luer connector and/or a needle or cannula capable of accessing a needle-free connector (NFC), a needle access connector (PRN), a luer access port (or female luer), or a septum such as, e.g., a BD NEXIVA™ needle septum.

The fluid transfer device 10 also includes a connector member 20 configured for selectively coupling the fluid transfer device 10 to, e.g., a needleless access connector of an adaptor coupled to the vascular access device. Connector member 20 is configured as an alligator clip-type connector, with opposing distal clip portions 21A, 21B allowing for securement of the fluid device 10 to a surface of the needleless access connector. A pair of proximal clip portions 22A, 22B are sized and configured to be pinched or otherwise manipulated by a user. Manipulation of the proximal clip portions 22A, 22B pivots the distal clip portions 21A, 21B away from one another, thereby resulting in release of the distal clip portions 21A, 21B from engagement with the needleless access connector. In some embodiments, the proximal clip portions 22A, 22B may include a textured surface and/or indicia so as to provide an indication to the user as to the functionality of the connector member 20

The fluid transfer device 10 further includes a locking member 28. As shown in FIGS. 2-6, the locking member 28 includes a locking arm 30 extending distally from a base portion 32, wherein the base portion 32 is configured to enable both securement of the locking member 28 to, e.g., a track extending along the introducer body 12 and selective longitudinal movement of the locking member along the introducer body 12.

Referring to FIG. 2, when the actuator 14 is in a first position substantially near the proximal end portion 18 of the introducer body 12, the probe 26 is retracted within the distal introducer portion 24 and the introducer body 12. Additionally, when the actuator 14 is in the first position, the locking member 28 is in a “disengaged” position along the introducer body 12, with the locking arm 30 being located proximally away from the connector member 20. In this position, the proximal clip portions 22A, 22B of the connector member 20 are capable of being pinched so as to allow for release the distal clip portions 21A, 21B from engagement with the needleless access connector and, thus, enable removal of the fluid transfer device 10 from engagement with the needleless access connector.

Conversely, referring to FIG. 3, when the actuator 14 is manipulated toward a second position at or near the distal end portion 16 of the introducer body 12, the probe 26 is advanced through (and distally beyond) the distal introducer portion 24. Additionally, advancement of the actuator 14 also advances the locking member 28 distally. When the actuator 14 is in its distal-most position along the introducer body 12, the locking arm 30 of locking member 28 is configured to be positioned substantially below at least one of the proximal clip portions 22A, 22B, with the locking arm 30 being sized and configured to substantially restrict inward motion of at least one of the proximal clip portions 22A, 22B when pinched. By restricting/blocking the inward motion of one or more of the proximal clip portions 22A, 22B, the user is unable to release the distal clip portions 21A, 21B from engagement with the needleless access connector and, thus, is unable to remove the fluid transfer device 10 from the needleless access connector when the flexible probe 26 is in the advanced position shown in FIG. 3.

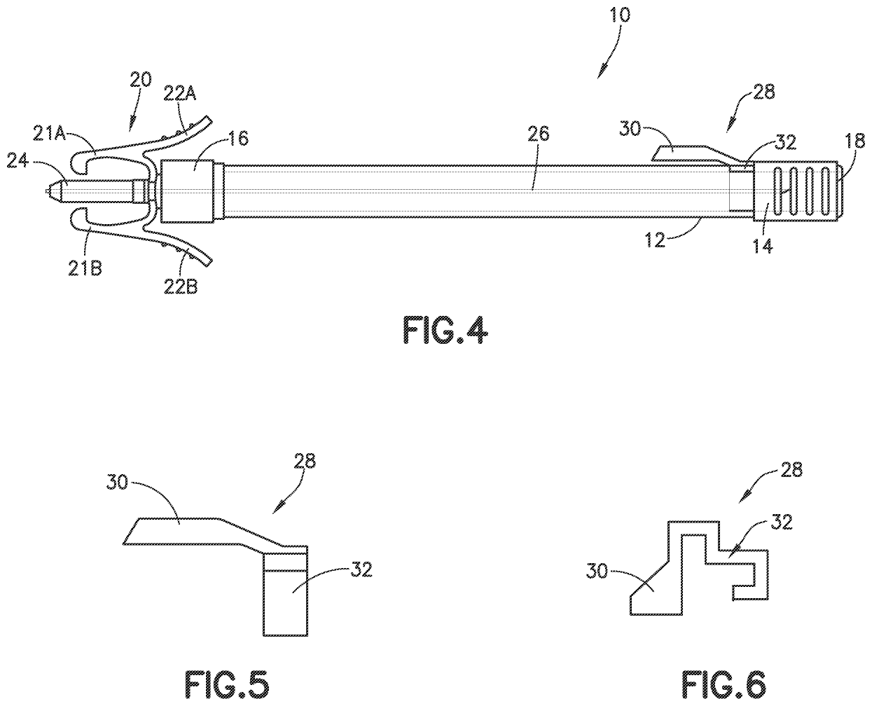

In some embodiments, the actuator 14 and at least the base portion 32 of the locking member 28 may be configured to engage or otherwise couple with one another upon contact between the actuator 14 and the locking member 28. For example, the actuator 14 and locking member 28 may couple by way of mechanical interference, magnetic connectors, or any other appropriate coupling means. In such embodiments, when the actuator 14 is manipulated proximally toward the first position to retract the flexible probe 26, the locking member 28 is simultaneously pulled proximally and away from the connector member 20, as is shown in FIG. 4. In this retracted position, the locking arm 30 positioned away from the connector member 20 such that inward movement of the proximal clip portions 22A, 22B is again possible, thereby allowing for the release of the distal clip portions 21A, 21B from engagement with the needleless access connector and, thus, enabling removal of the fluid transfer device 10 from the needleless access connector when the flexible probe 26 is at or near a retracted position. In this way, premature removal of the fluid transfer device 10 from the needleless access connector can be substantially avoided.

In other embodiments, the actuator 14 and the locking member 28 may not be configured to engage or otherwise couple upon contact between the actuator 14 and the locking member 28. In such embodiments, the user may be required to manually retract the locking member 28 in a proximal direction after the actuator 14 is returned to the first (i.e., proximal-most) position to retract the probe 26. Additionally and/or alternatively, in some embodiments, the locking member 28 may be manually displaced toward the connector 20 by the user prior to distal displacement of the actuator 14 to advance the probe 26 from the distal introducer portion 24. In such embodiments, the locking member 28 is configured to prevent premature removal of the fluid transfer device 10 from the needleless access connector as the probe 26 is being advanced.

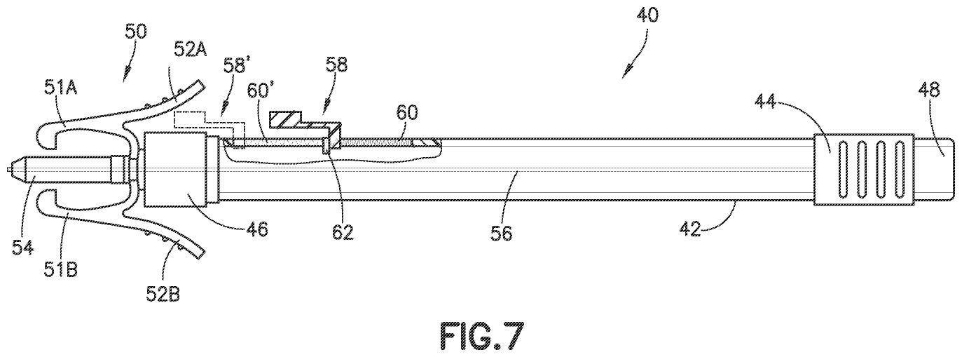

Next, referring to FIG. 7, a fluid transfer device 40 in accordance with another aspect of the present disclosure is shown. The fluid transfer device 40 includes an introducer body 42 and an actuator 44. The actuator 44 is configured to be linearly displaceable by a user along a track or other feature of the introducer body 42 between a proximal end portion 48 and a distal end portion 46 of the introducer body 42. The actuator 44 is operably coupled to an elongated, flexible probe 56 in order to selectively advance and retract the probe 56 through a distal introducer portion 54 located near the distal end portion 46 of the introducer body 42. While not shown in FIG. 7, it is to be understood that displacement of the actuator 44 in first direction advances the probe 56 through the distal introducer portion 54 such that the probe 56 can enter the patient's vasculature when fluid transfer device 40 is coupled to a vascular access device. Conversely, displacement of the actuator 54 in a second, opposite direction retracts the probe 56 through the distal introducer portion 54 to remove the probe 56 from the patient's vasculature.

The fluid transfer device 40 also includes a connector member 50 configured for selectively coupling the fluid transfer device 40 to, e.g., a needleless access connector of an adaptor coupled to the vascular access device. Connector member 50 is configured as an alligator clip-type connector, with opposing distal clip portions 51A, 51B allowing for securement of the fluid device 40 to a surface of the needleless access connector. A pair of proximal clip portions 52A, 52B are sized and configured to be pinched or otherwise manipulated by a user. Manipulation of the proximal clip portions 52A, 52B pivots the distal clip portions 51A, 51B away from one another, thereby resulting in release of the distal clip portions 51A, 51B from engagement with the needleless access connector.

The fluid transfer device 40 further includes a locking member 58. The locking member 58 is operably coupled to a biasing member 60 (e.g., a spring), with the biasing member 60 configured to selectively bias the locking member 58 in a distal direction.

Referring still to FIG. 7, when the actuator 44 is in a first position substantially near the proximal end portion 48 of the introducer body 42, the probe 56 is retracted within the distal introducer portion 54 and the introducer body 42. Additionally, when the actuator 44 is in the first position, the locking member 58 is in a “disengaged” position along the introducer body 42, with the locking member 58 being retained by a holding tab 62 such that the biasing member 60 is in a compressed state and the locking member 58 is held away from the connector member 50. In this position, the proximal clip portions 52A, 52B of the connector member 50 are capable of being pinched so as to allow for release the distal clip portions 51A, 51B from engagement with the needleless access connector and, thus, enable removal of the fluid transfer device 40 from engagement with the needleless access connector.

On the other hand, when the actuator 44 is manually slid toward a second position at or near the distal end portion 46 of the introducer body 42, the probe 56 is advanced through (and distally beyond) the distal introducer portion 54. Additionally, advancement of the actuator 44 also acts to release engagement between the holding tab 62 and the locking member 58. Such a release of the locking member 58 results in the biasing member 60 forcing the locking member 58 in a distal direction toward the connector member 50. Thus, as the actuator 44 is moved toward its distal-most position along the introducer body 42 to advance the probe 56, the locking member 58 and biasing member 60 (annotated as 58′ and 60′, respectively, in FIG. 7) are configured to position the locking member 58 substantially below at least one of the proximal clip portions 52A, 52B to substantially restrict inward motion of at least one of the proximal clip portions 52A, 52B when pinched. By restricting/blocking the inward motion of one or more of the proximal clip portions 52A, 52B, the user is unable to release the distal clip portions 51A, 51B from engagement with the needleless access connector and, thus, is unable to remove the fluid transfer device 40 from the needleless access connector when the flexible probe 56 is in an advanced position.

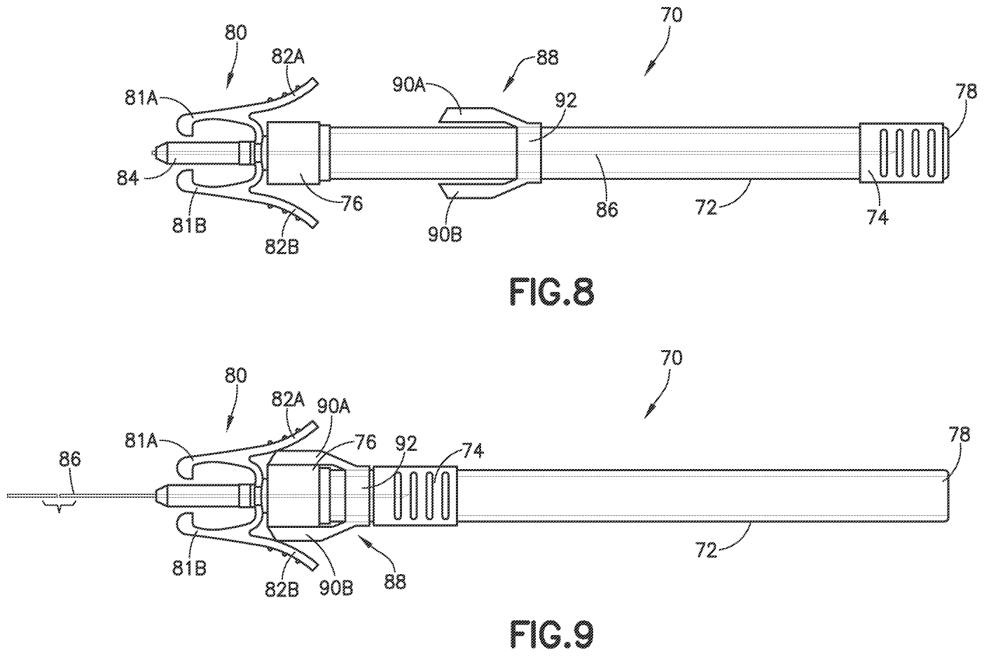

Referring now to FIGS. 8-10, a fluid transfer device 70 in accordance with another aspect of the present disclosure is illustrated. While fluid transfer device 10 shown and described above with respect to FIGS. 2-4 includes a locking member having only a single arm member configured to block actuation of a single proximal clip portion of the connector member, fluid transfer device 70 includes a locking member having a pair of arm members configured to block actuation of both proximal clip portions, as will be described in further detail herein.

The fluid transfer device 70 includes an introducer body 72 and an actuator 74. The actuator 74 is configured to be linearly displaceable by a user along a track or other feature of the introducer body 72 between a proximal end portion 78 and a distal end portion 76 of the introducer body 72. The actuator 74 is operably coupled to an elongated, flexible probe 86 to advance and retract the probe 86 through a distal introducer portion 84 located near the distal end portion 76 of the introducer body 72. Thus, displacement of the actuator 74 in first direction advances the probe 86 through the distal introducer portion 84 such that the probe 86 can enter the patient's vasculature when fluid transfer device 70 is coupled to a vascular access device. Conversely, displacement of the actuator 74 in a second, opposite direction retracts the probe 86 through the distal introducer portion 84 to remove the probe 86 from the patient's vasculature.

The fluid transfer device 70 includes a connector member 80 configured for selectively coupling the fluid transfer device 70 to, e.g., a needleless access connector of an adaptor coupled to the vascular access device. Connector member 80 is configured as an alligator clip-type connector, with opposing distal clip portions 81A, 81B allowing for securement of the fluid device 70 to a surface of the needleless access connector. A pair of proximal clip portions 82A, 82B are sized and configured to be pinched or otherwise manipulated by a user. Pinching of the proximal clip portions 82A, 82B pivots the distal clip portions 81A, 81B away from one another, thereby resulting in release of the distal clip portions 81A, 81B from engagement with the needleless access connector.

The fluid transfer device 70 further includes a locking member 88. As shown in FIGS. 8-11, the locking member 88 includes a pair of opposed locking arms 90A, 90B extending distally from a base portion 92. Referring to FIG. 11, a coupling member 94 also extends from the base portion 92 to enable both securement of the locking member 88 to, e.g., a track extending along the introducer body 72 and selective longitudinal movement of the locking member 88 along the introducer body 72.

Referring to FIG. 8, when the actuator 74 is in a first position substantially near the proximal end portion 78 of the introducer body 72, the probe 86 is retracted within the distal introducer portion 844 and the introducer body 72. Additionally, when the actuator 74 is in the first position, the locking member 88 is in a “disengaged” position along the introducer body 72, with the pair of opposed locking arms 90A, 90B being located proximally away from the connector member 80. In this position, the proximal clip portions 82A, 82B of the connector member 80 are capable of being pinched so as to allow for release the distal clip portions 81A, 81B from engagement with the needleless access connector and, thus, enable removal of the fluid transfer device 70 from engagement with the needleless access connector.

Conversely, referring to FIG. 9, when the actuator 74 is manipulated toward a second position at or near the distal end portion 76 of the introducer body 72, the probe 86 is advanced through (and distally beyond) the distal introducer portion 84. Additionally, advancement of the actuator 74 also advances the locking member 88 in a distal direction. When the actuator 74 is in its distal-most position along the introducer body 72, the pair of opposed locking arms 90A, 90B of locking member 88 are configured to be positioned substantially below respective proximal clip portions 82A, 82B, with the opposed locking arms 90A, 90B being sized and configured to substantially restrict inward motion of at least one of the proximal clip portions 82A, 82B when pinched. By restricting/blocking the inward motion of one or more of the proximal clip portions 82A, 82B, the user is unable to release the distal clip portions 81A, 81B from engagement with the needleless access connector and, thus, is unable to remove the fluid transfer device 70 from the needleless access connector when the flexible probe 86 is in the advanced position shown in FIG. 9.

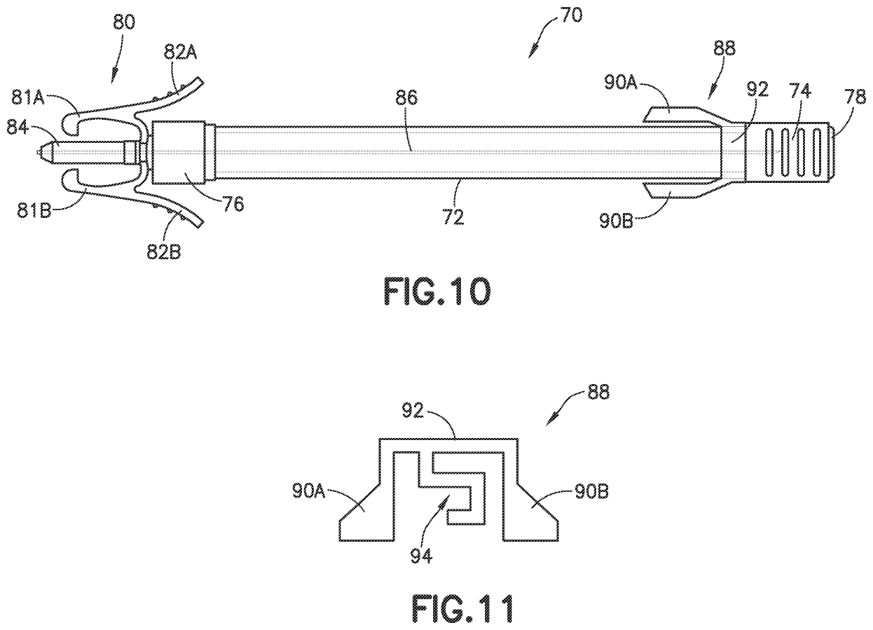

In some embodiments, the actuator 74 and at least the base portion 92 of the locking member 88 may be configured to engage or otherwise couple with one another upon contact between the actuator 74 and the locking member 88. For example, the actuator 74 and locking member 88 may couple by way of mechanical interference, magnetic connectors, or any other appropriate coupling means. In such embodiments, when the actuator 74 is slid in a proximal direction toward the first position to retract the flexible probe 86, the locking member 88 is simultaneously pulled proximally and away from the connector member 80, as is shown in FIG. 10. In this retracted position, the opposed locking arms 90A, 90B are positioned away from the connector member 80 such that inward movement of the proximal clip portions 82A, 82B is again possible, thereby allowing for the release of the distal clip portions 81A, 81B from engagement with the needleless access connector and, thus, enabling removal of the fluid transfer device 70 from the needleless access connector when the flexible probe 86 is at or near a retracted position. In this way, premature removal of the fluid transfer device 70 from the needleless access connector can be prevented.

Next, referring to FIGS. 12-14, a fluid transfer device 100 in accordance with another aspect of the present disclosure is illustrated. The fluid transfer device 100 includes an introducer body 102 and an actuator 104. The actuator 104 is configured to be linearly displaceable by a user along a track or other feature of the introducer body 102 between a proximal end portion 108 and a distal end portion 106 of the introducer body 102. The actuator 104 is operably coupled to an elongated, flexible probe 116 to advance and retract the probe 116 through a distal introducer portion 114 located near the distal end portion 106 of the introducer body 102. In other words, displacement of the actuator 104 in first direction advances the probe 116 through the distal introducer portion 114 such that the probe 116 is able to enter the patient's vasculature when fluid transfer device 100 is coupled to a vascular access device. On the other hand, displacement of the actuator 104 in a second, opposite direction retracts the probe 116 through the distal introducer portion 114, ultimately removing the probe 116 from the patient's vasculature.

The fluid transfer device 100 also includes a connector member 110 configured for selectively coupling the fluid transfer device 100 to, e.g., a needleless access connector of an adaptor coupled to the vascular access device. Connector member 110 is configured as an alligator clip-type connector, with opposing distal clip portions 111A, 111B allowing for securement of the fluid device 100 to a surface of the needleless access connector. A pair of proximal clip portions 112A, 112B are sized and configured to be pinched by a user. Manipulation of the proximal clip portions 112A, 112B pivots the distal clip portions 111A, 111B away from one another, thereby resulting in release of the distal clip portions 111A, 111B from engagement with the needleless access connector.

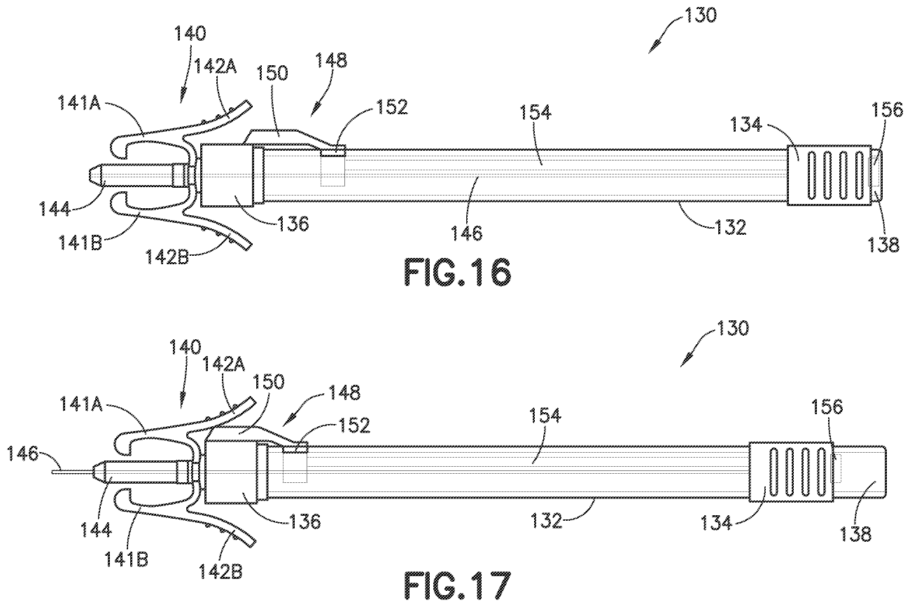

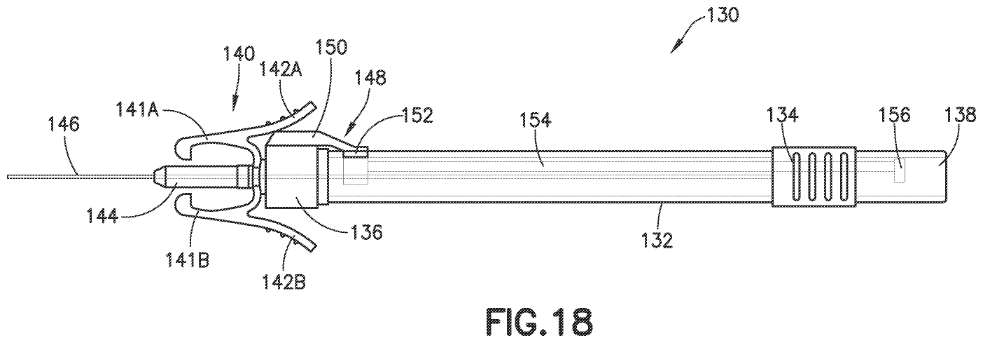

Additionally, the fluid transfer device 100 includes a locking member 118. As shown in FIGS. 12-15, the locking member 118 comprises a locking arm 120 extending distally from a base portion 122, wherein the base portion 122 is configured to enable both securement of the locking member 118 to, e.g., a track extending along the introducer body 102 and selective longitudinal movement of the locking member along the introducer body 102. While only a single locking arm 120 is shown in FIGS. 12-15, it is to be understood that a pair of opposing locking arms may be utilized, similar to those shown and described with respect to FIGS. 8-11.

The locking member 118 also includes an elongated tether portion 124 and a proximal tether member 126. As will be described in further detail below, the tether portion 124 and proximal tether member 126 act to control the timing of retraction of the locking arm 120 away from the connector member 110 during concurrent retraction of the actuator 104.

Referring to FIG. 12, when the actuator 104 is in a first position substantially near the proximal end portion 108 of the introducer body 102, the probe 116 is retracted within the distal introducer portion 114 and the introducer body 102. Additionally, when the actuator 104 is in the first position, the locking member 118 is in a “disengaged” position along the introducer body 102, with the locking arm 120 being located proximally away from the connector member 110. In this position, the proximal clip portions 112A, 112B of the connector member 110 are capable of being pinched to enable the release the distal clip portions 111A, 111B from engagement with the needleless access connector and, thus, enable removal of the fluid transfer device 100 from engagement with the needleless access connector.

On the other hand, referring to FIG. 13, when the actuator 104 is manually slid toward a second position at or near the distal end portion 106 of the introducer body 102, the probe 116 is advanced through (and distally beyond) the distal introducer portion 114. Advancement of the actuator 104 in this fashion also advances the locking member 118 distally, as the actuator 104 is configured to contact at least the base portion 122 of the locking member 118 to simultaneously slide the locking member 118 in a distal direction as the actuator 104 is being slid toward the distal end portion 106. Such movement of the base portion 122 and arm member 120 also results in corresponding distal movement of the elongated tether portion 124 and proximal tether member 126, as shown in FIG. 13.

When the actuator 104 is in its distal-most position along the introducer body 102, the locking arm 120 of locking member 118 is configured to be positioned substantially below at least one of the proximal clip portions 112A, 112B, with the locking arm 120 being sized and configured to substantially restrict inward motion of at least one of the proximal clip portions 112A, 112B when pinched. By restricting/blocking the inward motion of one or more of the proximal clip portions 112A, 112B, the user is unable to release the distal clip portions 111A, 111B from engagement with the needleless access connector and, thus, is unable to remove the fluid transfer device 100 from the needleless access connector when the flexible probe 116 is in the advanced position shown in FIG. 13.

In the embodiment shown in FIGS. 12-14, when the actuator 104 is manipulated proximally toward the first position to retract the flexible probe 116, the locking member 118 is not immediately pulled proximally and away from the connector member 110. Instead, the actuator 104 travels the length of the elongated tether portion 124 until it contacts the proximal tether portion 126, at which point the locking member 118 also begins to retract proximally away from the connector member 110. In this way, the elongated tether portion 124 acts to time disengagement of the locking member 118 from its position below the connector member 110, further ensuring that the proximal clip portions 112A, 112B cannot be pinched to remove the fluid transfer device 100 before the flexible probe 116 is fully retracted. The length of the elongated tether portion 124 can be varied and/or optimized based on desired engagement/disengagement of the locking member 118 relative to the connector member 110. For example, if the elongated tether portion 124 is only slightly shorter than the introducer body 102, the locking member 118 will stay “engaged” below the connector member 110 until the actuator 104 is nearly at its full proximal retraction point on the introducer body 102, thereby ensuring that the flexible probe 116 is fully (or close to fully) retracted prior to the connector member 110 being functional.

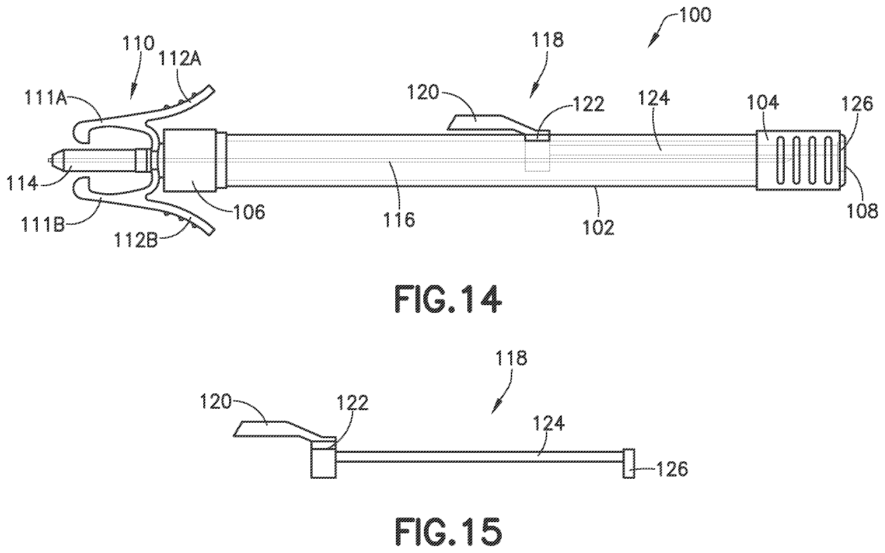

FIG. 14 illustrates both the actuator 104 and the locking member 118 in a fully-retracted position after use of the fluid transfer device. In this retracted position, the locking arm 120 is positioned away from the connector member 110 such that inward movement of the proximal clip portions 112A, 112B is again possible, thereby allowing for the release of the distal clip portions 111A, 111B from engagement with the needleless access connector and, thus, enabling removal of the fluid transfer device 100 from the needleless access connector when the flexible probe 116 is at or near a retracted position. In this way, premature removal of the fluid transfer device 100 from the needleless access connector can be prevented.

Referring now to FIGS. 16-18, a fluid transfer device 130 in accordance with another aspect of the present disclosure is illustrated. The fluid transfer device 130 includes an introducer body 132 and an actuator 134. The actuator 134 is configured to be linearly displaceable by a user along a track or other feature of the introducer body 132 between a proximal end portion 138 and a distal end portion 136 of the introducer body 132. The actuator 134 is operably coupled to an elongated, flexible probe 146 to advance and retract the probe 146 through a distal introducer portion 144 located near the distal end portion 136 of the introducer body 132.

The fluid transfer device 130 also includes a connector member 140 configured for selectively coupling the fluid transfer device 130 to, e.g., a needleless access connector of an adaptor coupled to the vascular access device. Connector member 140 is configured as an alligator clip-type connector, with opposing distal clip portions 141A, 141B allowing for securement of the fluid device 130 to a surface of the needleless access connector. A pair of proximal clip portions 142A, 142B are sized and configured to be pinched by a user. Such pinching of the proximal clip portions 142A, 142B pivots the distal clip portions 141A, 141B away from one another, thereby resulting in release of the distal clip portions 141A, 141B from engagement with the needleless access connector.

Additionally, the fluid transfer device 130 includes a locking member 148. The locking member 148 includes at least one locking arm 150 extending distally from a base portion 152, wherein the base portion 152 is configured to enable both securement of the locking member 148 to, e.g., a track extending along the introducer body 132 and selective longitudinal movement of the locking member along the introducer body 132. While only a single locking arm 150 is shown in FIGS. 16-18, it is to be understood that a pair of opposing locking arms may be utilized, similar to those shown and described with respect to FIGS. 8-11.

The locking member 148 also includes an elongated tether portion 154 and a proximal tether member 156. In the embodiment shown in FIGS. 16-18, the elongated tether portion 154 extends a substantial length of the elongated body 132. As will be described in further detail below, the tether portion 154 and proximal tether member 156 act to control the timing of advancement and retraction of the locking arm 150 to and away from the connector member 140 during concurrent movement of the actuator 134.

Referring to FIG. 16, when the actuator 134 is in a first position substantially near the proximal end portion 138 of the introducer body 132, the probe 146 is retracted within the distal introducer portion 144 and the introducer body 132. Additionally, when the actuator 144 is in the first position, the locking member 148 is in a “disengaged” position along the introducer body 132, with the locking arm 150 being located proximally away from the connector member 140. In this position, the proximal clip portions 142A, 142B of the connector member 140 are capable of being pinched to enable the release the distal clip portions 141A, 141B from engagement with the needleless access connector and, thus, enable removal of the fluid transfer device 130 from engagement with the needleless access connector.

Conversely, referring to FIG. 17, when the actuator 134 is manually slid toward a second position at or near the distal end portion 136 of the introducer body 132, the probe 146 is advanced through (and distally beyond) the distal introducer portion 144. Advancement of the actuator 134 in this fashion also advances the locking member 148 distally, as the actuator 134 is configured to have a slight engagement with the elongated tether portion 154 and/or the proximal tether member 156 so as to simultaneously slide the locking member 148 in a distal direction as the actuator 134 is being slid toward the distal end portion 136. It is to be understood that engagement between the actuator 134 and one or more parts of the locking member 148 may be any appropriate, releasable engagement such as, e.g., interference engagement, mechanical engagement, magnetic engagement, etc.

When the actuator 134 is initially moved toward the distal end portion 136 along the introducer body 132, the engagement between the actuator 134 and the locking member 148 causes the locking arm 150 to move to a position substantially below at least one of the proximal clip portions 142A, 142B, with the locking arm 150 being sized and configured to substantially restrict inward motion of at least one of the proximal clip portions 142A, 142B when pinched. By restricting/blocking the inward motion of one or more of the proximal clip portions 142A, 142B, the user is unable to release the distal clip portions 141A, 141B from engagement with the needleless access connector and, thus, is unable to remove the fluid transfer device 130 from the needleless access connector when the flexible probe 146 is advanced through the distal introducer portion 144, as shown in FIG. 17.

Referring to FIG. 18, after the locking member 148 is in its “engaged” position below the connector member 140, the actuator 134 may continue to be moved distally so as to advance the probe 146 beyond the distal introducer portion 144 and into the patient's vasculature. Furthermore, while not shown, when the actuator 134 is moved proximally toward the first position to retract the flexible probe 146, the locking member 148 is not immediately pulled proximally and away from the connector member 140. Instead, the actuator 134 travels the length of the elongated tether portion 154 until it contacts the proximal tether portion 156, at which point the locking member 148 also begins to retract proximally away from the connector member 140. In this way, the elongated tether portion 154 acts to time disengagement of the locking member 148 from its position below the connector member 140, ensuring that the proximal clip portions 142A, 142B cannot be pinched to remove the fluid transfer device 130 before the flexible probe 146 is fully retracted. The length of the elongated tether portion 154 can be varied and/or optimized based on desired engagement/disengagement of the locking member 148 relative to the connector member 140. For example, in the embodiment shown in FIGS. 16-18, the elongated tether portion 154 is only slightly shorter than the introducer body 132, enabling the locking member 148 to remain in a “locking” position below the connector member 140 until the actuator 134 is nearly at its full proximal retraction point on the introducer body 132, thereby ensuring that the flexible probe 146 is fully (or close to fully) retracted prior to the connector member 140 being functional for release of the fluid transfer device 130 from the needleless access connector.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A fluid transfer device for delivery of a probe to a patient's vascular system, the fluid transfer device comprising:
an introducer body;
a distal introducer portion positioned at a distal end portion of the introducer body and configured to penetrate an access connector of a vascular access device;
an actuator, wherein the actuator is linearly displaceable along at least a portion of the introducer body, and wherein the actuator is operably coupled to the probe to advance and retract the probe through the distal introducer portion based on direction of displacement of the actuator;
a connector member positioned proximate the distal introducer portion and configured for releasable connection to at least one surface of the access connector, wherein the connector member comprises a pair of opposing distal clip portions and a pair of opposing proximal clip portions; and
a locking member, wherein the locking member is linearly displaceable along at least a portion of the introducer body and is configured to prevent actuation of at least one of the pair of opposing proximal clip portions when the actuator is displaced at least partially toward first position and to allow actuation of at least one of the pair of opposing proximal clip portions when the actuator is displaced at least partially toward a second position.

2. The fluid transfer device of claim 1, wherein the locking member comprises a single locking arm member, and wherein the single locking arm member is configured to block actuation of a single one of the pair of opposing proximal clip portions of the connector member.

3. The fluid transfer device of claim 2, wherein the single locking arm member extends distally from a base portion, and wherein the base portion is configured to be slidable along at least a portion of the introducer body.

4. The fluid transfer device of claim 1, wherein the locking member comprises a pair of opposing arm members, and wherein the pair of opposing arm members are configured to block actuation of the pair of opposing proximal clip portions of the connector member.

5. The fluid transfer device of claim 4, wherein the pair of opposing arm members extend distally from a base portion, and wherein the base portion is configured to be slidable along at least a portion of the introducer body.

6. The fluid transfer device of claim 5, wherein the actuator is releasably engagable with the base portion.

7. The fluid transfer device of claim 1, wherein the locking member comprises a biasing member configured to bias the locking member in one direction.

8. The fluid transfer device of claim 7, further comprising a holding tab, wherein the holding tab is configured to hold the locking member proximally away from the connector member.

9. The fluid transfer device of claim 8, wherein the holding tab is configured to be displaced by the actuator upon contact with the actuator, and wherein displacement of the holding tab causes distal displacement of the locking member toward the second position under force of the biasing member.

10. The fluid transfer device of claim 1, wherein the locking member comprises at least one arm member and an elongated tether portion extending proximally from the at least one arm member, wherein the at least one arm member is configured to block actuation of at least one of the pair of opposing proximal clip portions of the connector member.

11. The fluid transfer device of claim 10, wherein the actuator is configured for releasable engagement with at least a portion of the elongated tether portion.

12. The fluid transfer device of claim 1, wherein the locking member is manually displaceable by a user.

13. A fluid transfer device for needle-free delivery of a probe to a patient's vascular system, the fluid transfer device comprising:
an introducer body having a distal end portion and a proximal end portion;
an actuator, wherein the actuator is linearly displaceable along at least a portion of the introducer body, and wherein the actuator is operably coupled to the probe to advance and retract the probe based on direction of displacement of the actuator;
a connector member positioned proximate the distal end portion of the introducer body and configured for releasable connection to at least one surface of an access connector; and
a locking member, wherein the locking member is configured to prevent disconnection of the connector member from the access connector when the actuator is advanced to a first position along the introducer body and to allow disconnection of the connector member when the actuator is retracted to a second position along the introducer body.

14. The fluid transfer device of claim 13, wherein the locking member comprises a single locking arm member, and wherein the single locking arm member is configured to block actuation of the connector member.

15. The fluid transfer device of claim 13, wherein the locking member comprises a pair of opposing arm members, and wherein the pair of opposing arm members are configured to block actuation of the connector member.

16. The fluid transfer device of claim 13, wherein the locking member comprises at least one arm member and an elongated tether portion extending proximally from the at least one arm member, wherein the at least one arm member is configured to block actuation of the connector member.

17. The fluid transfer device of claim 16, wherein the actuator is configured for releasable engagement with at least a portion of the elongated tether portion.

18. A fluid transfer device for needle-free delivery of a probe to a patient's vascular system, the fluid transfer device comprising:
an introducer body having a distal end portion and a proximal end portion;
an actuator, wherein the actuator is linearly displaceable along at least a portion of the introducer body, and wherein the actuator is operably coupled to the probe to advance and retract the probe based on direction of displacement of the actuator;
a connector member positioned proximate the distal end portion of the introducer body and configured for releasable connection to at least one surface of an access connector; and
a locking member, wherein the locking member comprises at least one arm member and an elongated tether portion extending proximally from the at least one arm member, wherein the at least one arm member is configured to selectively block actuation of the connector member based upon the position of the actuator along the introducer body.

19. The fluid transfer device of claim 18, wherein the actuator is configured for releasable engagement with at least a portion of the elongated tether portion.

20. The fluid transfer device of claim 18, wherein the connector member comprises a pair of opposing distal clip portions and a pair of opposing proximal clip portions, and further wherein the at least one arm member of the locking member is configured to block actuation of at least one of the pair of opposing proximal clip portions.
